(19) United States
(12) Patent Application Publication
(10) Pub. No.: US 2024/0086633 A1
(43) Pub. Date: Mar. 14, 2024

(54) METHOD, SYSTEM AND NON-TRANSITORY STORAGE MEDIUM FOR GENERATING AND OUTPUTTING A MESSAGE

(71) Applicant: 9 Rise International Mobile Health Technology Co., LTD., New Taipei City (TW)

(72) Inventors: Yi-Ru CHIU, New Taipei City (TW); Ting-Yi LI, New Taipei City (TW); Hong-Xun WANG, New Taipei City (TW); Jin-Lin CHEN, New Taipei City (TW); Chih-Hsuan YEH, New Taipei City (TW); Chia-Chi YIN, New Taipei City (TW); Wei-Ting LI, New Taipei City (TW); Po-Lun CHANG, New Taipei City (TW)

(21) Appl. No.: 18/306,889

(22) Filed: Apr. 25, 2023

(30) Foreign Application Priority Data
Sep. 13, 2022 (TW) 111134544

(51) Int. Cl. G06F 40/186

(52) U.S. Cl. CPC G06F 40/186

(57) ABSTRACT

A method for generating and outputting a message is implemented using an electronic device the stores a computer program product and a text database. The text database includes a main message template, a template text that includes a placeholder, and a word group that includes a plurality of preset words for replacing the placeholder. The method includes: in response to receipt of a command for execution of the computer program product, displaying an editing interface including the main message template; in response to receipt of user operation of a selection of the main message template, displaying the template text; in response to receipt of user operation of a selection of one of the preset words via the user interface, generating an edited text by replacing the placeholder with the one of the preset words in the template text; and outputting the edited text as a message.

### CROSS-REFERENCE TO RELATED APPLICATION

This application claims priority to Taiwanese Invention Patent Application No. 111134544, filed on Sep. 13, 2022.

### FIELD

The disclosure relates to a method, a system and a non-transitory storage medium for generating and outputting a message.

### BACKGROUND

In the field of home care services, a service provider may be dispatched to a residence of a client (e.g., a patient, an elder, etc.) to provide supportive care or medical care. In the event of an emergency, the service provider needs to contact an associate of the client (e.g., a family of the client, a medical personnel, etc.) to report the situation. Typically, the reporting may be done using a telephone call or a text message.

It is noted that, in the case where a text message is used, to adequately include the condition of the client in the message in an emergency may be difficult. Additionally, since the content of the text message may not always reflect the actual condition of the client (because of the nature of the emergency), the receiver of the text message may need more communication with the service provider to get the whole picture and make the appropriate response.

### SUMMARY

Therefore, one object of the disclosure is to provide a method for generating and outputting a message.

According to one embodiment of the disclosure, the method is implemented using an electronic device that includes a processor, a data storage medium, a user interface and a display. The data storage medium stores a computer program product and a text database. The text database includes at least one main message template, at least one template text that is associated with the at least one main message template and that includes at least one placeholder, and at least one word group that is associated with the placeholder and that includes a plurality of preset words at least one of which is to be selected for replacing the placeholder. The method includes:
a) in response to receipt of a command via the user interface for execution of the computer program product, controlling the display to display an editing interface, the editing interface including the at least one main message template;
b) in response to receipt of user operation of a selection of one of the at least one main message template via the user interface, controlling the display to display one of the at least one template text that is associated with said one of the at least one main message template;
c) in response to receipt of user operation of a selection of one of the preset words via the user interface, generating an edited text by replacing a corresponding one of the at least one placeholder with the one of the preset words thus selected in the template text; and
d) outputting the edited text as a message.

Another object of the disclosure is to provide an electronic device that is configured to implement the above-mentioned method.

According to one embodiment of the disclosure, the electronic device includes electronic device for generating and outputting a message includes:
a processor;
a data storage medium connected to the processor, the data storage medium storing a computer program product and a text database therein, the text database including at least one main message template, at least one template text that is associated with the at least one main message template and that includes at least one placeholder, and at least one word group that is associated with the placeholder and that includes a plurality of preset words at least one of which is to be selected for replacing the placeholder; and
a user interface and a display connected to the processor.

The processor is configured to:
in response to receipt of a command via the user interface for execution of the computer program product, control the display to display an editing interface, the editing interface including the at least one main message template;
in response to receipt of user operation of a selection of one of the at least one main message template via the user interface, control the display to display the at least one template text;
in response to receipt of user operation of a selection of one of the preset words via the user interface, generate an edited text by replacing one of the at least one placeholder that is associated with the one of the at least one main message template with the one of the preset words thus selected in the template text; and
output the edited text as a message.

Another object of the disclosure is to provide a non-transitory storage medium storing a computer program product therein. The computer program product includes instructions that, when executed by a processor of an electronic device, cause the processor to perform steps of the above-mentioned method.

### BRIEF DESCRIPTION OF THE DRAWINGS

Other features and advantages of the disclosure will become apparent in the following detailed description of the embodiments with reference to the accompanying drawings. It is noted that various features may not be drawn to scale.

FIG. 1 is a block diagram illustrating an electronic device for generating and outputting a message according to one embodiment of the disclosure.

FIG. 2 illustrates an exemplary editing interface that is in the form of a graphic user interface (GUI) and that is to be displayed on a display according to one embodiment of the disclosure.

FIGS. 3A, 3B and 3C illustrate an exemplary text database according to one embodiment of the disclosure.

FIG. 4 is a flow chart illustrating steps of a method for generating and outputting a message according to one embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

Throughout the disclosure, the term “coupled to” or “connected to” may refer to a direct connection among a plurality of electrical apparatus/devices/equipment via an electrically conductive material (e.g., an electrical wire), or an indirect connection between two electrical apparatus/devices/equipment via another one or more apparatus/devices/equipment, or wireless communication.

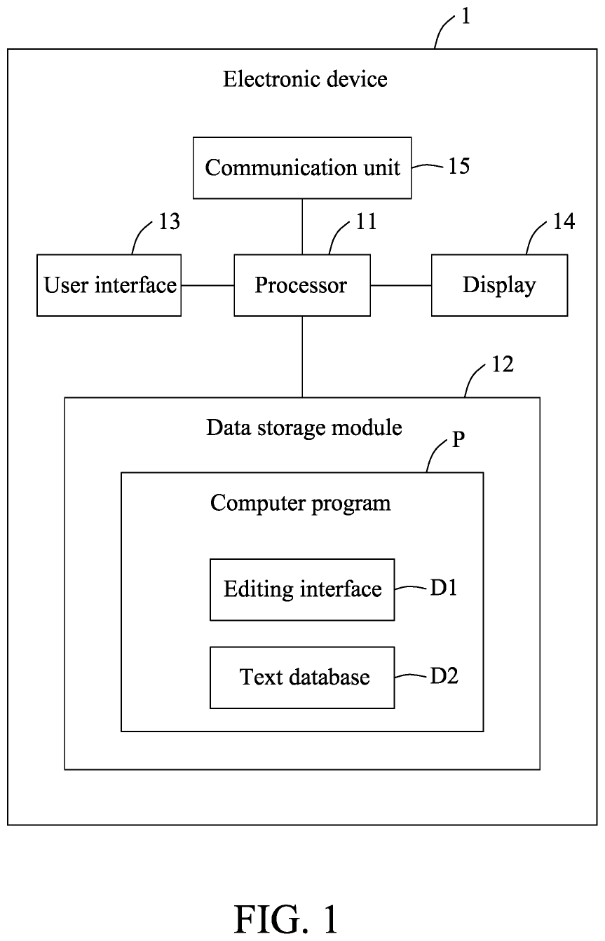

FIG. 1 is a block diagram illustrating an electronic device 1 for generating and outputting a message according to one embodiment of the disclosure. In the embodiment, the electronic device 1 may be embodied using a portable electronic device (e.g., a smart phone, a laptop, a tablet, etc.) or a personal computer, and includes a processor 11, a data storage module 12, a user interface 13, a display 14, and a communication unit 15.

The processor 11 may include, but not limited to, a single core processor, a multi-core processor, a dual-core mobile processor, a microprocessor, a microcontroller, a digital signal processor (DSP), a field-programmable gate array (FPGA), an application specific integrated circuit (ASIC), and/or a radio-frequency integrated circuit (RFIC), etc.

The data storage module 12 is connected to the processor 11, and may be embodied using, for example, random access memory (RAM), read only memory (ROM), programmable ROM (PROM), firmware, and/or flash memory, etc. The data storage module 12 is configured to store a computer program P. The computer program P includes instructions that, when executed by the processor 11, cause the processor 11 to perform the operations as described below.

The user interface 13 and the display 14 are connected to the processor 11. In this embodiment, the user interface 13 and the display 14 may be embodied using a touchscreen built-in the electronic device 1. In other embodiments, the user interface 13 may be embodied using a keyboard, a mouse, a writing pad, etc., and the display 14 may be a component that is separate from the user interface 13.

The communication unit 15 is connected to the processor 11, and may include one or more of a radio-frequency integrated circuit (RFIC), a short-range wireless communication module supporting a short-range wireless communication network using a wireless technology of Bluetooth® and/or Wi-Fi, etc., and a mobile communication module supporting telecommunication using Long-Term Evolution (LTE), the third generation (3G), the fourth generation (4G) or fifth generation (5G) of wireless mobile telecommunications technology, or the like.

In use, the electronic device 1 may be held by a service provider that is dispatched to a residence of a client (e.g., a patient, an elder, etc.) to provide supportive care or medical care. Prior to going to the residence, the service provider may operate the electronic device 1 to download the computer program P via the communication unit 15, and install the computer program P. In this embodiment, when the computer program P is downloaded, a text database D2 is also downloaded and stored in the data storage module 12.

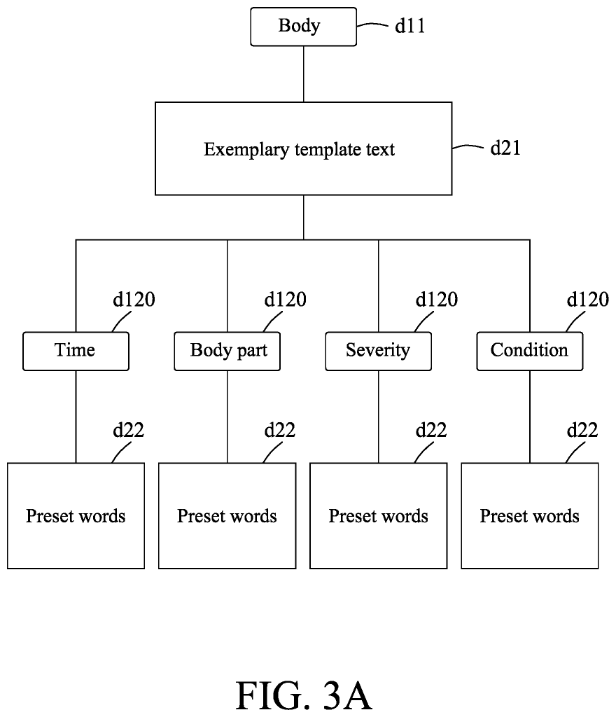

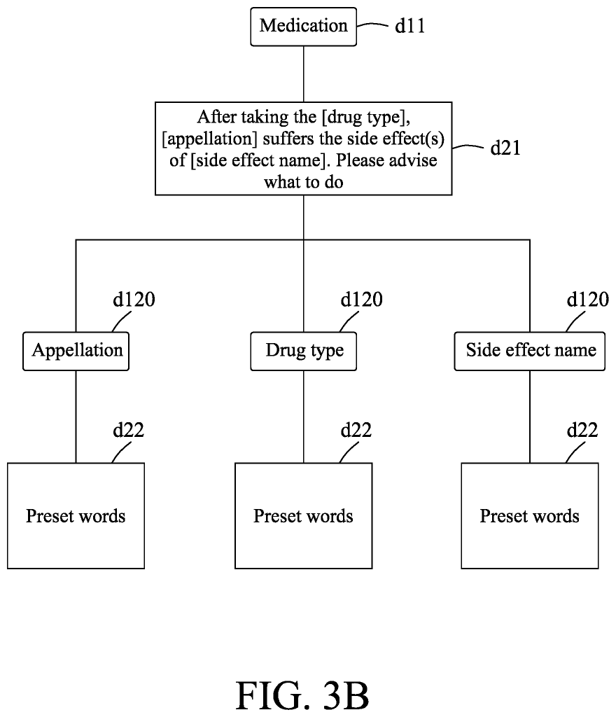

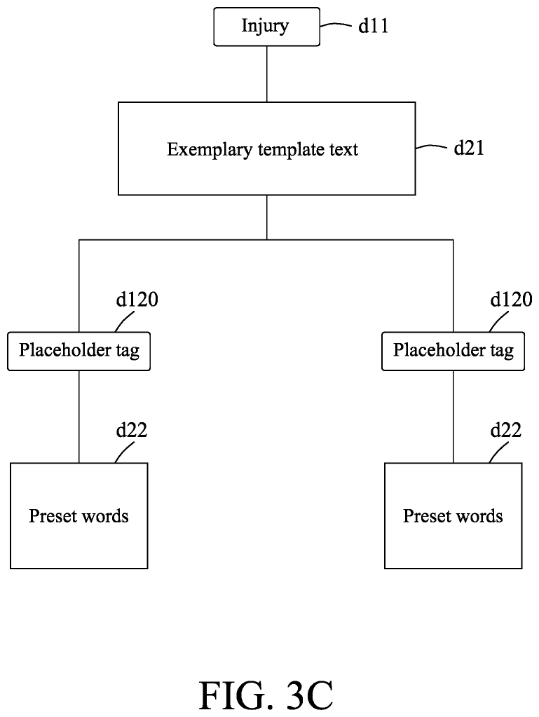

FIGS. 3A, 3B and 3C illustrate an exemplary text database D2 according to one embodiment of the disclosure. In this embodiment, the text database D2 includes at least one main message template d11, at least one template text d21 that is associated with the at least one main message template d11, a plurality of placeholder tags d120 each being associated with the template text d21, a plurality of word groups d22 each being associated with a respective one of the placeholder tags d120, and a plurality of preset words d220 each included in one of the word groups d22.

Specifically, in this embodiment, the text database D2 includes three main message templates d11 (that are labeled “body,” “medication,” “injury,” respectively), and three template texts d21 that are associated with the main message templates d11, respectively. An exemplary template text d21 associated with the main message template d11 labeled “medication” may read “After taking the [drug type], [appellation] suffers the side effect(s) of [side effect name]. Please advise what to do.” Accordingly, such a text message includes three placeholders each indicating a blank section in the template text d21 to be filled, namely, an appellation of the client, a name of the drug used, and a side effect. Each of the placeholders is associated with a placeholder tag d120, and therefore three placeholder tags d120 are present for the main message template d11 labeled “medication.” In this embodiment, for one of the other two main message templates d11 (labeled “body”), the associated template text d21 may read “At [time], the [body part] suffers a [severity] [condition]. Please advise what to do.” Such a template text d21 includes four placeholders, and correspondingly, four placeholder tags d120 are present. For the other one of the other two main message templates d11 (labeled “injury”), the associated template text d21 may include two placeholders, and correspondingly, two placeholder tags d120 are present. Therefore, a total of nine placeholder tags d120 are present in the text database D2. It is noted that, in other embodiments, each of the template texts d21 may include other numbers of placeholders.

For each of the placeholder tags d120, an associated word group d22 is present. That is to say, in the embodiment of FIGS. 3A, 3B and 3C, a total of nine word groups d22 are present in the text database D2. Each of the word groups d22 includes a plurality of preset words d220 that are used for helping to replace a specific placeholder included in the corresponding template text d21. For example, in the above exemplary template text d21 associated with the main message template d11 labeled “medication,” “After taking the [drug type], [appellation] suffers the side effect(s) of [side effect name]. Please advise what to do,” one of the placeholder tags d120 is labeled “drug type,” and the corresponding word group d22 may include ten preset words d220, each naming a kind of drug that may be taken by the client (e.g., “antipyretic,” “painkiller,” “antimalarial drug,” “antibiotic,” “bactericide,” “stimulant,” “sedative,” “mood stabilizer,” “antihypertensive,” and “diabetes medication”). In other embodiments, the preset words d220 may be in the form of one or more characters, each being a Chinese character, an English character, a number, a symbol, etc.

It is noted that in other embodiments, different numbers of the main message templates d11 may be present, each having content and an associated template text d21 that may be similar to or different from those illustrated in the embodiment of FIGS. 3A, 3B and 3C. Additionally, with respect to each of the main message templates d11, the template text d21 may have content and a number of placeholders different from those exemplarily illustrated in the embodiment of FIGS. 3A, 3B and 3C. In other words, with respect to each of the template texts d21, different numbers of placeholder tags d120 may be present. Each of the placeholder tags d120 is associated with a word group d22 that may include preset words d220 which may be similar to or different from those illustrated in the embodiment of FIGS. 3A, 3B and 3C. That is to say, the content of the text database D2 is not limited to what has been described or shown in FIGS. 3A, 3B and 3C.

In some embodiments, depending on the condition of the client (which may be provided by an associate of the client or obtained from a medical record of the client), the content of the text database D2 may be customized for the client. In other words, in the text database D2, the main message templates d11, the template texts d21, the placeholder tags d120, and the preset words d220 included in the word groups d22 may be edited, added or deleted by users based on needs. In some embodiments, the computer program P may be associated with multiple text databases D2, each being customized for a specific client.

During the course of providing services, in the event of an emergency, the service provider needs to contact the associate of the client (e.g., a family of the client, a medical personnel, etc.) to report the situation. Typically, the reporting may be done using a telephone call or a text message.

In this embodiment, the service provider holding the electronic device 1 may execute the computer program P, so as to initiate a message generating process. In response, the processor 11 generates an editing interface D1 and controls the display 14 to display the editing interface D1.

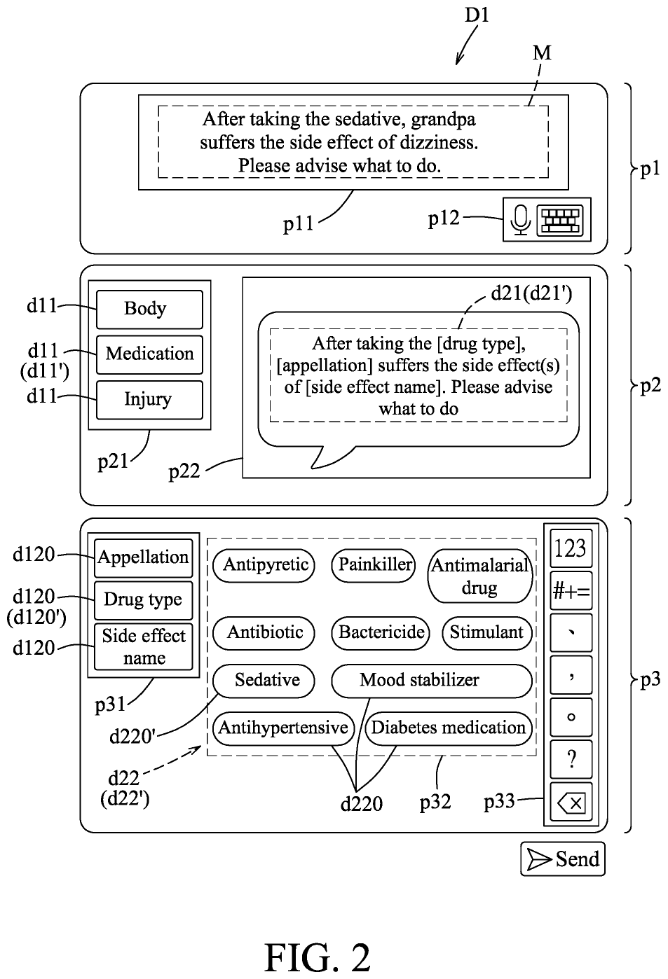

FIG. 2 illustrates an exemplary editing interface D1 that is in the form of a graphic user interface (GUI) and that is to be displayed on the display 14 according to one embodiment of the disclosure. In this embodiment, the editing interface D1 includes a message preview section p1, a message template selection section p2 and a word selection section p3. While in this embodiment, the message preview section p1, the message template selection section p2 and the word selection section p3 are simultaneously displayed on the display 14 in a manner as illustrated in FIG. 2, in other embodiments, the editing interface D1 may be displayed in other manners, and is not limited to the manner of FIG. 2.

The message preview section p1 includes a message zone p11 that displays an edited text M, and an editing zone p12 that includes one or more editing selections (e.g., a voice input button, a virtual keyboard, etc.). The message template selection section p2 includes a first zone p21 that displays at least one main message template d11 to be selected, and a second zone p22 that displays a selected template text d21′ that is associated with the main message template d11 which is selected. It is noted that while in this embodiment, three different main message templates d11 are present, in other embodiments, additional main message template(s) d11 may be present, or alternatively, only one or two main message templates d11 may be present.

In response to selection of the at least one main message template d11, the second zone p22 displays the associated template text d21. In the example of FIG. 2, the main message template d11 labeled “medication” is selected (therefore labeled as a selected message template d11′), and so the second zone p22 displays the associated template text d21 (therefore labeled as the selected template text d21′).

The word selection section p3 includes a first zone p31 that displays a plurality of placeholder tags d120, and a second zone p32 that displays a word group d22 that is associated with a selected placeholder tags d120′ (therefore labeled as a selected word group d22′). In the embodiment of FIG. 3B, the placeholder tags d120 labeled “drug type” is selected, and the selected word group d22′ that is associated with the selected placeholder tag d120′ and that includes the preset words d220 as described above are displayed. It is noted that while in this embodiment, three different placeholder tags d120 are present, in other embodiments, additional placeholder tags d120 may be present, or alternatively, only one or two placeholder tags d120 may be present.

In some embodiments, the word selection section p3 may include a third zone p33 that includes a number of input buttons for editing the message (e.g., adding/deleting punctuation marks, numbers, etc.).

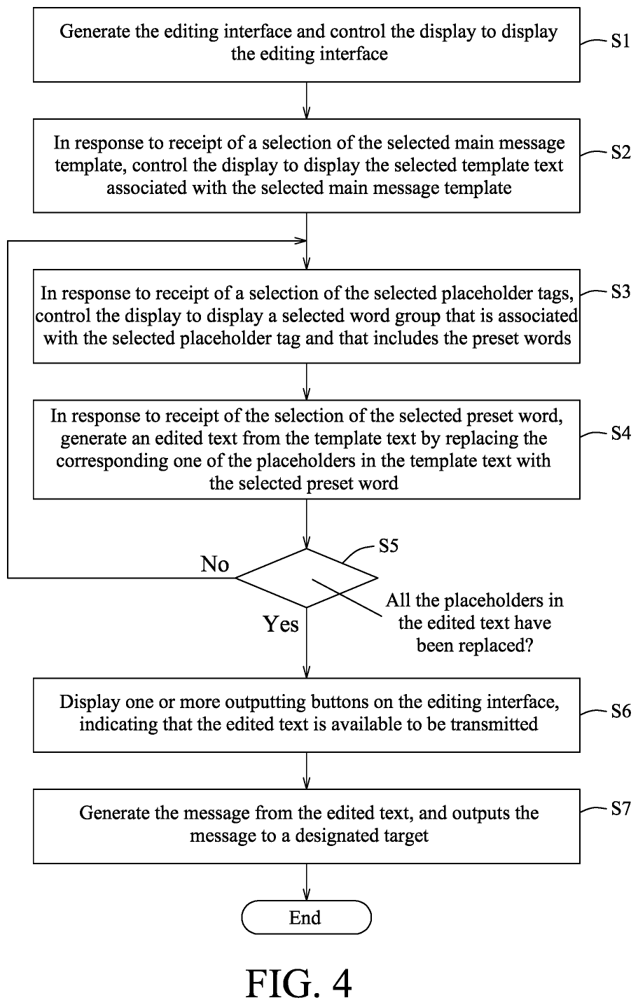

FIG. 4 is a flow chart illustrating steps of a method for generating and outputting a message according to one embodiment of the disclosure. In this embodiment, the method is implemented using the electronic device 1 that is as shown in FIG. 1, and that is installed with the computer program P thereon.

The method may be initiated by the service provider who holds the electronic device 1, and who may be aware that an emergency situation had occurred (e.g., the client is feeling unwell, is injured or is suffering side effects due to medication, etc.). In such a situation, the service provider may operate the user interface 13 of the electronic device 1 to transmit a user command to the processor 11 to execute the computer program P.

In response, in step S1, the processor 11 generates the editing interface D1 and controls the display 14 to display the editing interface D1. In this embodiment, at least the first zone p21 of the message template selection section p2 is displayed at this stage, but is not limited to such. That is to say, the three main message templates d11 are displayed, and the service provider is able to operate the user interface 13 to select one of the three main message templates d11 as a selected main message template d11′. In this embodiment, the service provider may touch a part of the display 14 where one of the three main message templates d11 is displayed, in order to select the one of the three main message templates d11 as the selected main message template d11′. In some embodiments where the electronic device 1 is a personal computer or a laptop where the display 14 is not a touch screen, the service provider may operate a keyboard of the user interface 13 to select the one of the three main message templates d11.

In response to receipt of a selection of the selected main message template d11′ via the user interface 13, in step S2, the processor 11 controls the display 14 to display the selected template text d21′ that is associated with the selected main message template d11′ in the second zone p22 of the message template selection section p2. In some embodiments, the selected template text d21′ is also displayed in the message zone p11 of the message preview section p1. In addition, the processor 11 controls the display 14 to display the plurality of placeholder tags d120 that are associated with the selected template text d21′ in the first zone p31 of the word selection section p3.

In the example of FIG. 2, the selected main message template d11′ is labeled “medication,” and the selected template text d21′ associated with the selected main message template d11′ is “After taking the [drug type], [appellation] suffers the side effect(s) of [side effect name]. Please advise what to do.” It is noted that in this embodiment, the content displayed in the message zone p11 of the message preview section p1 is available for the service provider to edit directly, and the content displayed in the second zone p22 of the message template selection section p2 is not available for the service provider to edit directly.

At this stage, the service provider is able to edit the selected template text d21′ by directly editing the content displayed in the message zone p11 of the message preview section p1, or selecting one of the placeholder tags d120 as the selected placeholder tag d120′ for designating a corresponding one of the placeholders to be replaced in the selected template text d21′.

In response to receipt of a selection of the selected placeholder tags d120′, in step S3, the processor 11 controls the display 14 to display a selected word group d22′ that is associated with the selected placeholder tag d120′ and that includes the preset words d220. In the embodiment of FIG. 2, the selected placeholder tag d120′ is “drug type,” and the associated selected word group d22′ includes ten preset words d220, each indicating one type of drug, is displayed on the second zone p32 of the word selection section p3. At this stage, the service provider is able to select one of the preset words d220 that fits the emergency situation, to replace the placeholder in the selected template text d21′ that is associated with the drug type. In the embodiment of FIG. 2, the preset word d220 “sedative” is selected, serving as a selected preset word d220′.

Then, in step S4, in response to receipt of the selection of the selected preset word d220′, the processor 11 proceeds to generate an edited text M from the template text d21′ by replacing the corresponding one of the placeholders in the template text d21′ with the selected preset word d220′, so as to reflect the selection. That is to say, the edited text M includes the selected preset word d220′. In the above example, the selected template text d21′ “After taking the [drug type], [appellation] suffers the side effect(s) of [side effect name]. Please advise what to do.” is edited in this step due to the service provider selecting the preset word d220′, and the resulting edited text M now reads “After taking the sedative, [appellation] suffers the side effect(s) of [side effect name]. Please advise what to do”, reflecting the corresponding one of the placeholders being replaced by an actual word. The edited text M is then displayed on the message zone p11 of the message preview section p1 and the second zone p22 of the message template selection section p2. It is noted that in some embodiments, the service provider may operate the user interface 13 to input one or more words to replace a specific placeholder in the case none of the preset words d220 contained in the word group is deemed adequate.

Afterwards, the service provider is enabled to select another one of the placeholder tags d120, so as to replace one of the remaining placeholders in the edited text M.

In step S5, the processor 11 determines whether all the placeholders in the edited text M have been replaced. In the case where all the placeholders of the edited text M have been replaced, the flow proceeds to step S6. Otherwise, in the case where at least one of the placeholders of the edited text M has not been replaced, the flow goes back to step S3 to wait for the service provider to select another one of the placeholder tags d120.

In this embodiment, the service provider selects another one of the placeholder tags d120 (e.g., the one labeled “appellation”) as the selected placeholder tag d120′. Then, the processor 11 controls the display 14 to display another word group d22 that is associated with the selected placeholder tag d120′ in the second zone p32 of the word selection section p3. This specific word group d22 may include a number of preset words d220, each indicating a specific appellation for the client, such as “grandpa,” “grandma,” “uncle,” “aunt,” “child,” etc. As such, the service provider is enabled to select one of the preset words d220 that fits the appellation of the client. In this embodiment, the client is referred to as a grandpa, and as such, the preset word d220 “grandpa” may be selected as the selected preset word d220′.

Then, the flow proceeds to step S4. In response to receipt of the selection of the selected preset word d220′, the processor 11 proceeds to generate another edited text M from the edited text M by replacing a corresponding one of the placeholders in the edited text M with the selected preset word d220′, so as to reflect the selection. In the above example, the edited text M “After taking the sedative, [appellation] suffers the side effect(s) of [side effect name]. Please advise what to do.” is edited in this step in view of the service provider's selection of the preset word d220′, and the resulting edited text M now reads “After taking the sedative, grandpa suffers the side effect(s) of [side effect name]. Please advise what to do.” The edited text M is then displayed on the message zone p11 of the message preview section p1 and the second zone p22 of the message template selection section p2.

The flow then proceeds to step S5, and since not all of the placeholders have been replaced, the determination result makes the flow go back to step S3.

At this stage, the service provider may select the remaining one of the placeholder tags d120 (e.g., the one labeled “side effect”) as the selected placeholder tag d120′. Then, the processor 11 controls the display 14 to display another word group d22 that is associated with the selected placeholder tag d120′ in the second zone p32 of the word selection section p3. This specific word group d22 (i.e., the selected word group d22′) may include a number of preset words d220, each indicating a specific side effect such as “dizziness,” “headache,” “fever,” “vomiting,” “coughing,” “shivering,” etc. As such, the service provider is enabled to select one of the preset words d220 that fits the side effect of the client. In this embodiment, the side effect may include dizziness, and as such, the preset word d220 “dizziness” may be selected as the selected preset word d220′.

Then, the flow proceeds to step S4. In response to receipt of the selection of the selected preset word d220′, the processor 11 proceeds to generate another edited text M from the edited text M by replacing one of the placeholders in the edited text M with the selected preset word d220′, so as to reflect the selection. In the above example, the edited text M “After taking the sedative, grandpa suffers the side effect(s) of [side effect name]. Please advise what to do.” is edited in this step because the service provider had selected the preset word d220′, and the resulting edited text M now reads “After taking the sedative, grandpa suffers the side effect of dizziness. Please advise what to do.” The edited text M is then displayed on the message zone p11 of the message preview section p1 and the second zone p22 of the message template selection section p2.

It is noted that in this case, multiple side effects might occur, and therefore, after selecting one preset word d220 (“dizziness”), the service provider may select one or more additional preset words d220 as the selected preset word(s) 220′. As a result, the processor 11 may further insert the additional selected preset word(s) d220′ into the edited text M. For example, in the case where the client is experiencing both dizziness and headache, the service provider may select the two corresponding preset words d220 as the selected preset words 220′, and the resulting edited text M may thus read “After taking the sedative, grandpa suffers the side effect(s) of dizziness and headache. Please advise what to do.” The edited text M is then displayed on the message zone p11 of the message preview section p1 and the second zone p22 of the message template selection section p2.

Then, the flow goes to step S5, and since all of the placeholders in the edited text M has now been replaced, the flow proceeds to step S6.

In step S6, the processor 11 controls the display 14 to display one or more outputting buttons on the editing interface D1, indicating that the edited text M is available to be transmitted. At this stage, the service provider is able to operate the user interface 13 to generate a user command for sending out the edited text M as a message. In response to receipt of the user command to output the message, in step S7, the processor 11 generates the message from the edited text M, and outputs the message to a designated target.

In the embodiment of FIG. 2, with the edited text M, a “send” button is shown. In other embodiments, the set of outputting buttons may include a “copy to clipboard” button, and in response to the service provider pressing the “copy to clipboard” button (i.e., the user command), the processor 11 outputs the edited text M as a message by storing the edited text M in a clipboard storage medium that serves as the designated target, enabling the service provider to paste the message into other applications such as a short message service (SMS), an instant messaging (IM) application (e.g., Whatsapp®, Line®, or other IM applications), etc.

In some embodiments, the set of outputting buttons may include a “send to . . . ” button, and in response to the service provider pressing the “send to . . . ” button (i.e., the user command), the processor 11 outputs the edited text M as a message by sending the edited text M to one or more pre-established contact(s) or a group chat that serves as the designated target via SMS or an IM application. In use, this is done by the processor 11 controlling the communication unit 15 to transmit the edited text M to a communication server (not depicted in the drawings) that is associated with the SMS or the IM application, such that the communication server is configured to transmit the edited text M as the message to the pre-established contact(s), to be displayed by one or more electronic devices held by the pre-established contact(s). This pre-established contact(s) or group chat may be established by the service provider prior to providing service.

It is noted that in the stage of step S6, the service provider is still able to edit the edited text M by selecting one of the main message templates d11 (which makes the flow go back to step S2), selecting one of the placeholder tags d120 (which makes the flow go back to step S3), or clicking on the editing zone p12 to add or delete words from the edited text M before proceeding to input the user command for transmitting the message.

As such, the edited text M may be generated and outputted quickly, so as to inform the contact of the emergency situation in a timely fashion.

To sum up, embodiments of the disclosure provide a method, an electronic device and a computer program product for generating and outputting a message. In the embodiments, a number of template texts d21 are prepared for use in advance, each including one or more placeholders that indicate blank sections for the service provider to fill in, and word groups for assisting the service provider in replacing each of the placeholders. By displaying a specifically designed editing interface D1, the service provider is enabled to simply select one of the preset words to replace one of the placeholders, in order to create an edited text that describes the situation of a client to which the service provider is providing health care or medical care service. In the manner as described above, the service provider may be able to complete the creation of the edited text without having to manually type a word. In this manner, the edited text may be quickly generated and outputted, especially in the cases where the medical condition of the client may necessitate usage of complex medical-related terms.

According to one embodiment of the disclosure, there is provided a non-transitory storage medium storing a computer program product therein (e.g., the aforementioned computer program P). The computer program product includes instructions that, when executed by a processor of an electronic device, cause the processor to perform steps of the method as described above and shown in FIG. 4.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for generating and outputting a message, the method being implemented using an electronic device that includes a processor, a data storage medium, a user interface and a display, the data storage medium storing a computer program product and a text database, the text database including at least one main message template, at least one template text that is associated with the at least one main message template and that includes at least one placeholder, and at least one word group that is associated with the placeholder and that includes a plurality of preset words at least one of which is to be selected for replacing the placeholder, the method comprising:
a) in response to receipt of a command via the user interface for execution of the computer program product, controlling the display to display an editing interface, the editing interface including the at least one main message template;
b) in response to receipt of user operation of a selection of one of the at least one main message template via the user interface, controlling the display to display one of the at least one template text that is associated with said one of the at least one main message template;
c) in response to receipt of user operation of a selection of one of the preset words via the user interface, generating an edited text by replacing a corresponding one of the at least one placeholder with the one of the preset words thus selected in the template text; and
d) outputting the edited text as a message.

2. The method as claimed in claim 1, the template text including a plurality of placeholders, the text database further including a plurality of placeholder tags associated with the plurality of placeholders, respectively, and a plurality of word groups associated with the plurality of placeholder tags, respectively, each of the word groups including a plurality of preset words at least one of which is to be selected for replacing one of the placeholders that is associated with the word group, wherein step c) includes:
controlling the display to display the plurality of placeholder tags in the editing interface;
in response to receipt of user operation of a selection of one of the placeholder tags via the user interface, displaying the plurality of preset words included in one of the word groups that is associated with the one of the placeholder tags in the editing interface;
in response to receipt of user operation of a selection of one of the preset words, generating the edited text by replacing one of the placeholders that is associated with the one of the placeholder tags with the one of the preset words thus selected in the template text.

3. The method as claimed in claim 2, wherein step c) includes:
in response to receipt of user operation of selecting multiple preset words among the preset words, generating the edited text by replacing the one of the placeholders with the multiple preset words in the template text.

4. The method as claimed in claim 1, wherein step d) includes transmitting the edited text to a communication server that is associated with a short message service (SMS) or an instant messaging (IM) application, to enable the communication server to transmit the edited text to at least one electronic device held by at least one pre-established contact for display thereon.

5. An electronic device for generating and outputting a message, comprising:
a processor;
a data storage medium connected to the processor, the data storage medium storing a computer program product and a text database therein, the text database including at least one main message template, at least one template text that is associated with the at least one main message template and that includes at least one placeholder, and at least one word group that is associated with the placeholder and that includes a plurality of preset words at least one of which is to be selected for replacing the placeholder; and
a user interface and a display connected to the processor, wherein the processor is configured to:
in response to receipt of a command via the user interface for execution of the computer program product, control the display to display an editing interface, the editing interface including the at least one main message template;
in response to receipt of user operation of a selection of one of the at least one main message template via the user interface, control the display to display the at least one template text;
in response to receipt of user operation of a selection of one of the preset words via the user interface, generate an edited text by replacing one of the at least one placeholder that is associated with the one of the at least one main message template with the one of the preset words thus selected in the template text; and
output the edited text as a message.

6. The electronic device as claimed in claim 5, the template text including a plurality of placeholders, the text database further including a plurality of placeholder tags associated with the plurality of placeholders, respectively, and a plurality of word groups associated with the plurality of placeholder tags, respectively, each of the word groups including a plurality of preset words at least one of which is to be selected for replacing one of the placeholders that is associated with the word group, wherein the processor controls the display to display the at least one template text by:
controlling the display to display the plurality of placeholder tags in the editing interface;
in response to receipt of user operation of a selection of one of the placeholder tags via the user interface, displaying the plurality of preset words included in one of the word groups that is associated with the one of the placeholder tags in the editing interface;
in response to receipt of user operation of a selection of one of the preset words, generating the edited text by replacing one of the placeholders that is associated with the one of the placeholder tags with the one of the preset words thus selected in the template text.

7. The electronic device as claimed in claim 6, wherein the processor further controls the display to display the at least one template text by:
in response to receipt of user operation of selecting multiple preset words among the preset words, generating the edited text by replacing the one of the placeholders with the multiple preset words in the template text.

8. The electronic device as claimed in claim 5, wherein the processor outputs the edited text by transmitting the edited text to a communication server that is associated with a short message service (SMS) or an instant messaging (IM) application, to enable the communication server to transmit the edited text to at least one electronic device held by at least one pre-established contact for display thereon.

9. A non-transitory storage medium storing a computer program product therein, the computer program product including instructions that, when executed by a processor of an electronic device, cause the processor to perform steps of a method as claimed in claim 1.
